(19) United States
(12) Patent Application Publication
(10) Pub. No.: US 2026/0000879 A1
(43) Pub. Date: Jan. 1, 2026

(54) MICRONEEDLE THERAPY DEVICE EQUIPPED WITH STACKING MODE

(71) Applicant: CURIOSIS INC., Seoul (KR)

(72) Inventors: Hoyoung YUN, Seoul (KR); Heedon AHN, Seoul (KR); Kichan KIM, Seoul (KR); Jihoon SHIN, Seoul (KR); Kyuntae KIM, Seoul (KR); Chihyoung SHIM, Seoul (KR)

(73) Assignee: CURIOSIS INC., Seoul (KR)

(21) Appl. No.: 19/303,701

(22) Filed: Aug. 19, 2025

Related U.S. Application Data
(63) Continuation of application No. PCT/KR2024/004407, filed on Apr. 4, 2024.

(30) Foreign Application Priority Data
Apr. 7, 2023 (KR) 10-2023-0046128
Aug. 9, 2023 (KR) 10-2023-0104315

(51) Int. Cl. A61M 37/00; A61M 5/32; A61M 39/24

(52) U.S. Cl. CPC A61M 37/0015; A61M 5/3202; A61M 39/24; A61M 2037/0023

(57) ABSTRACT

The present invention relates to a microneedle therapy system and, more particularly, to a microneedle therapy device and system equipped with a stacking mode which allows needles to be stacked repeatedly with a single button click and enables injecting an active ingredient into multiple layers of the skin so as to be treated at once.

### CROSS-REFERENCE TO RELATED APPLICATIONS

This application is a continuation of International Application No. PCT/KR2024/004407 filed on Apr. 4, 2024, which claims priority to Korean Patent Application No. 10-2023-0046128 filed on Apr. 7, 2023, and Korean Patent Application No. 10-2023-0104315 filed on Aug. 9, 2023, the entire contents of which are herein incorporated by reference.

### TECHNICAL FIELD

The present disclosure relates to a microneedle therapy device and system, and more particularly to a microneedle therapy device and system equipped with a stacking mode in which a needling stacking is repeatedly performed by pressing a one-shot button, and effective ingredients are injected to several layers of a skin to treat the skin at once.

### BACKGROUND

In general, a health condition of a skin has a large impact on the appearance. Thus, in recent years, various methods have been developed for the purpose of lightening the skin, improving wrinkles, moisturizing the skin, increasing resilience of the skin, and the like, in addition to the purpose of treating a skin disease.

Further, as is well known, the human skin may be roughly classified into an epidermal layer (epidermis), a dermal layer (dermis) and a fat layer.

Among these, the dermal layer occupying a substantial portion of the skin is composed of a papillary layer and a plexiform layer. Capillaries, lymphatic tubes and the like are located in the papillary layer. Collagens as collagen fibers associated with wrinkles in the skin, elastin as elastic fibers that give elasticity to the skin, a substrate and the like are contained in the plexiform layer.

As described above, a visual condition of the skin is greatly dependent on a health status of the dermal layer. Accordingly, various methods implemented for improving the condition of the skin are often targeted for the dermal layer.

Since the dermal layer is protected by the epidermal layer as described above, when pharmacological agents to be delivered to the dermal layer are applied onto the skin, an amount of the pharmacological agents which reach the dermal layer and a reaching speed thereof may be drastically reduced.

Accordingly, in order to rapidly deliver the pharmacological agents applied onto the epidermal layer to the dermal layer, a method of applying pressure or ultrasonic vibrations has been proposed. This method has the advantage of not damaging the epidermal layer but requires a part for generating the pressure or the ultrasonic vibrations. This may cause an increase in cost and size.

As an approach for solving such matters, a wound treatment efficacy using fine needles has been disclosed in 1995, and a method (microneedle therapy system “MTS”) for forming passages in the epidermal layer with microneedles and delivering pharmacological agents, which induce a desired efficacy, to the dermal layer, has been widely used.

The MTS uses the microneedles having a thickness of 0.2 millimeter (mm) or less and a length of 2 mm or less to physically damage the epidermal layer and the dermal layer. This stimulates fibroblasts to induce production of collagens. Further, the MTS injects pharmacological agents such as vitamins, hyaluronic acids or the like to be sufficiently adsorbed to the epidermal layer and the dermal layer, thereby improving a treatment efficacy. As a technology for improving such a treatment efficacy, a number of documents including the following documents in the related art have been disclosed.

On the other hand, in a microneedle therapy system in the related art, moving (needling) microneedles to penetrate a skin includes (1) an operation of moving the microneedles when a user presses a head of a piston and (2) an operation of moving the microneedles by a motor.

However, the operating using the piston is a burdensome tack because the user needs to repeatedly press the head to repeat the needling. In addition, the operation using the motor is a burdensome tack because the user needs to repeatedly press an operation start button in a manual manner to perform the needling a multiple number of times and operate the motor.

### DOCUMENTS IN RELATED ART

(Patent Document 1) Korean Patent Application Registration No. 10-0922138 (2009 Oct. 9.)
(Patent Document 2) Korean Patent Application Registration No. 10-2206623 (2021 Jan. 18.)
(Patent Document 3) Korean Patent Application Registration No. 10-2147856 (2020 Aug. 19.)

### SUMMARY

### Technical Object

The present disclosure was made to solve the above-mentioned matters, and the present disclosure is for the purpose of providing a microneedle therapy device and system equipped with a stacking mode in which a needling is repeatedly performed by pressing a one-shot button, and effective ingredients are injected to several layers of a skin to treat the skin at once.

### Means for Solving Object

According to an example embodiment of the present disclosure, a microneedle therapy device may include a memory storing one or more instructions, and at least one processor configured to execute the one or more instructions stored in the memory to control a needling in which a microneedle moves forward and backward at a home position and returns to the home position. The at least one processor may be configured to execute the one or more instructions to receive user input information about the microneedle therapy device, which is input by a user, and performs a stacking mode based on the user input information, and the stacking mode may repeatedly perform a stacking set in which a needling stacking is consecutively performed two or more times at a same or different time intervals at a same position on a skin.

According to an example embodiment of the present disclosure, the needling stacking in the stacking mode may be repeatedly performed twice to 10 times. Further, the needling stacking in the stacking mode may be performed at needle penetration depths which are identical to or different from each other.

According to an example embodiment of the present disclosure, each of the needle penetration depths may be in a range of more than 0 to 7 millimeter (mm) or less. The needling stacking in the stacking mode may be repeatedly performed twice or three times. The needle penetration depths may be identical to or different from each other in a range of 0.25 to 6 mm.

According to an example embodiment of the present disclosure, the stacking mode may be performed in a manual mode or an automatic mode. In the manual mode, the user input information may be required each time when a first stacking set performed at a first skin position proceeds to a second stacking set performed at a second skin position. In the automatic mode, the first stacking set may automatically proceed to the second stacking set based on a first round of user input information initially input by the user. The microneedle may have a hollow or solid structure.

According to an example embodiment of the present disclosure, the microneedle therapy device may further include a needle cover, a pressure generation mechanism, a valve configured to be open and closed to supply a compressed gas from the pressure generation mechanism inward of the needle cover, and a microneedle arranged inside the needle cover and configured to move forward or backward via an opening provided in the needle cover. The at least one processor may control the opening and closing of the valve and the movement of the microneedle, and may control a valve open time point at which the valve is opened based on the user input information.

In an aspect, the pressure generation mechanism may be configured to generate the compressed gas and supply the compressed gas inward of the needle cover.

According to an example embodiment of the present disclosure, the at least one processor may control a power of the pressure generation mechanism based on the user input information. Further, the valve may be configured to be open while the microneedle penetrates the skin. Further, the valve is configured to be open while the microneedle is withdrawn from the skin after reaching a final penetration depth. Further, the valve may be configured to be open for a predetermined period of time after the microneedle is completely withdrawn from a skin. The user input information may be information about a penetration depth at which the microneedle penetrates the skin at the valve open time point the user wants.

According to an example embodiment of the present disclosure, a microneedle therapy device may include a memory storing one or more instructions, and at least one processor configured to execute the one or more instructions stored in the memory such that a needling in which a microneedle moves forward and backward at a home position and returns to the home position is performed in an automatic mode. In the automatic mode, a first needling may automatically proceed to a second needling based on a first round of user input information initially input by the user.

A microneedle therapy system according to another example embodiment of the present disclosure may include the aforementioned microneedle therapy device, and a display device configured to communicate with the at least one processor of the microneedle therapy device, configured to provide a user interface for receiving setting information about an operation of the microneedle therapy device from the user, and configured to transmit the setting information input by the user to the at least one processor of the microneedle therapy device.

According to an example embodiment of the present disclosure, the user interface may include a user interface for setting a positive-pressure generation time point for each needling. Further, the user interface may include a user interface for setting a penetration depth of the microneedle when the positive pressure is generated at the positive-pressure generation time point.

According to yet another example embodiment of the present disclosure, a method for injecting effective ingredients into a skin using the aforementioned microneedle therapy device may include setting an opening time of the valve based on the user input information, operating the pressure generation mechanism to generate a pneumatic pressure in a predetermined inner space of the microneedle therapy device; and moving the microneedle arranged inside the needle cover forward and backward via the opening of the needle cover. In the act of moving the microneedle, the valve provided between the predetermined inner space and the needle cover may be configured to be open based on the valve open time set so that the pneumatic pressure is applied into the needle cover.

According to an example embodiment of the present disclosure, the microneedle therapy device used for the method for injecting the effective ingredients into the skin may have a design or configuration described above or below in detail.

### Effect of the Invention

According to an example embodiment of the present disclosure, by automatically repeating a needling operation at a predetermined time interval, it is possible to eliminate a burdensome tack that requires a user to press a needling start button every time.

Further, according to an example embodiment of the present disclosure, by repeatedly performing a single operation mode in which penetration depths of microneedles are set to be different from each other for each needling in a predetermined consecutive plurality of needling stackings, it is possible to treat several layers of a skin (for example, a papillary layer, a plexiform layer and the like) at once.

Further, according to an example embodiment of the present disclosure, by setting positive-pressure generation time points for each needling in the aforementioned single operation mode to be different from each other, it is possible to optimize a profile in which effective ingredients are injected to each layer of the skin to be treated.

Further, according to an example embodiment of the present disclosure, a sub-cision effect by microneedles may be increased by the needling stacking. Thus, the present disclosure may be useful in treating scars or the like on the skin.

### BRIEF DESCRIPTION OF THE DRAWINGS

FIG. 1 is a schematic view of a microneedle therapy system according to an example embodiment of the present disclosure.

FIG. 2 is an exemplary perspective view showing an appearance of a microneedle therapy device according to the present disclosure.

FIG. 3 is an example of a user interface for receiving information about a time interval of needling operations from a user.

FIG. 4 is an example of a user interface for setting a stacking mode.

FIG. 5 is another example of the user interface for setting the stacking mode.

FIG. 6 illustrates a sequence of an operation of a pneumatic pump and opening/closing control of a solenoid valve according to an example embodiment of the present disclosure.

FIG. 7A shows a state in which the final penetration depth is 4 mm and a microneedle according to an example embodiment of the present disclosure moves forward by 3 mm, and FIG. 7B shows a state in which the microneedle according to an example embodiment of the present disclosure moves backward by 1 mm after penetrating by 4 mm which is a final penetration depth.

FIG. 8 is an exemplary cutaway cross-sectional view of a portion of the microneedle therapy device of FIG. 2.

FIG. 9 is an exemplary cross-sectional view of a microneedle unit which constitutes the microneedle therapy device according to an example embodiment of the present disclosure.

FIGS. 10A and 10B are explanatory views illustrating a front end of a needle cover which constitutes the microneedle unit of FIG. 9, and a modified example thereof.

FIG. 11 is a schematic view illustrating an example in which effective ingredients are injected using hollow microneedles constituting the microneedle unit according to an example embodiment of the present disclosure.

FIG. 12 is an explanatory view illustrating an example in which the microneedle therapy device including the microneedles according to an example embodiment of the present disclosure is used.

FIGS. 13A, 13B, 14A, and 14B are explanatory views illustrating examples in which the microneedles constituting the microneedle unit according to the present disclosure are formed in a hollow structure and a solid structure, respectively.

FIG. 15 is a flowchart for explaining a method of injecting effective ingredients into a skin using the microneedle therapy device according to an example embodiment of the present disclosure.

### DETAILED DESCRIPTION

Example embodiments of the present disclosure will be described in detail with reference to the accompanying drawings for the clarification of the technical sprit of the present disclosure. Here, in the description of the present disclosure, when it is determined that a detailed description of a related known function or configuration may unnecessarily obscure the gist of the present disclosure, the detailed description thereof will be omitted. In the drawings, constituent elements that are substantially the same in configuration will be denoted by the same reference numeral and symbols as possible even if they are displayed on the other drawings. For the sake of convenience in description, both an apparatus and a method will be described if necessary. Operations described in the present disclosure may not be necessarily performed in a sequence described herein but may be performed in a parallel manner, a selective manner or an individual manner.

Terms used in example embodiments are general terms that are currently widely used while their respective functions in the present disclosure are taken into consideration. However, the terms may be changed depending on the intention of one of ordinary skilled in the art, legal precedents, emergence of new technologies, and the like. Also, in particular cases, terms that are arbitrarily selected by the applicant of the present disclosure may be used, and in this case, the meanings of these terms may be described in detail in the corresponding disclosure. Accordingly, the terms used herein should be defined based on the meanings thereof and the content throughout the specification, instead of a simple name of the term.

Throughout the present disclosure, expressions in the singular form should be understood to encompass expressions in the plural form unless the context clearly indicates otherwise. The term “includes”, “has” or the like are intended to include features, numeric characters, processes, operations, constituent elements, parts, or a combination thereof, and should be understood not to exclude one or more other features, numeric characters, processes, operations, constituent elements, parts, or a combination thereof, or additional features and the like. That is, when a part “comprise or includes” a constituent element through the specification, this means that the part may further include other constituent elements, rather than excluding other constituent elements, unless other stated.

The expression such as “at least one” encompass the entire list of components and does not individually represent respective constituent elements in the list. For example, the expression “at least one of A, B, and C” may include the following meanings: “A alone”, “B alone”, “C alone”, “both A and B together”, “both A and C together”, “both Band C together”, or “all three of A, B, and C together”.

In addition, the terms such as “part,” “- - -er, - - -or,” “module” and the like used in the present disclosure may refer to a unit that performs at least one function or operation, which may be realized as hardware or software, or may be realized as a combination of hardware and software.

Through the present disclosure, when one constituent element is referred to as being “connected” to another constituent element, the one constituent element may be “directly connected” to another constituent element, or may be “indirectly connected” or “electrically connected” to another constituent element by intervening yet another constituent element therebetween. Further, when a part “comprise or includes” a constituent element through the specification, this means that the part may further include other constituent elements, rather than excluding other constituent elements, unless other stated.

The expression “configured (or set) to- - -” described herein may be alternatively expressed to, for example, “suitable for- - -,” “having the capacity to- - -,” “designed to- - -,” “adapted to- - -,” “made to- - -,” “capable of- - -,” or the like depending on situations. The expression “configured (or set) to- - -” may not necessarily mean only “specifically designed to- - -” in hardware. Alternatively, in some circumstances, the expression “system configured to- - -” may mean that the system is “capable of- - -” together with other apparatus(es) or constituent element(s). The phrase “processor configured (or set) to perform A, B and C” may mean, for example, a dedicated processor (for example, embedded process) configured to perform a respective operation, or a generic-purpose processor (for example, a central processing unit (CPU) or an application processor) capable of performing respective operations by executing one or more software programs stored in a memory.

Although the terms including ordinal numbers such as a first, a second and the like used herein may be used to describe various constituent elements, such constituent elements should not be limited by terms including the ordinal numbers. The above terms may be used to distinguish a constituent element from another constituent element in a description of the specification. For example, a first constituent element may be named as a second constituent element in another description of the specification without departing from the scope of the present disclosure. Similarly, the second constituent element may be named as the first constituent element in another description of the specification. The term “and/or” may be used to represent a combination of a plurality of related items described herein or at least one of the plurality of related items.

The term “about” used in the present disclosure may refer to within 10% of a given value or range, specifically within 5%, more specifically within 1%.

Hereinafter, preferred example embodiments according to the present disclosure will be described in more detail with reference to the accompanying drawings.

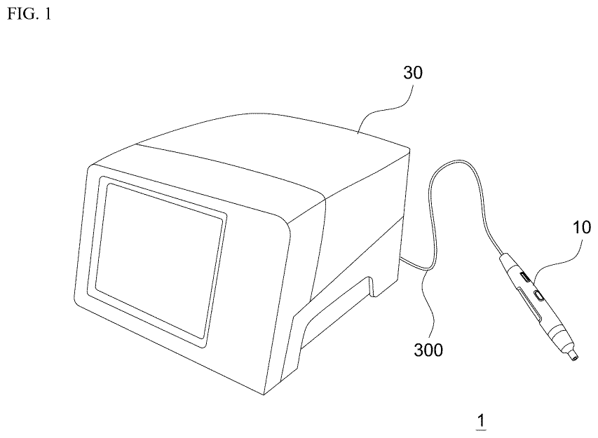

FIG. 1 is a schematic view of a microneedle therapy system 1 according to an example embodiment of the present disclosure.

A microneedle therapy device 10 according to an example embodiment of the present disclosure has a configuration in which microneedles are linearly moved by a rotary motor to perform a needling operation rather than a configuration in which the microneedles are moved to perform the needling operation when a user presses a head of a piston structure. The needling operation is controlled by a controller. As an example for implementing this, as illustrated in FIG. 1, the microneedle therapy system 1 according to an example embodiment of the present disclosure may include the microneedle therapy device 10, a display device 30, and a cable 300 connecting both.

The display device 30 provides a user interface for receiving setting information about the operation of the microneedle therapy device 10, which is input by the user, and transmits the setting information input by the user to the controller of the microneedle therapy device 10 via the cable 300.

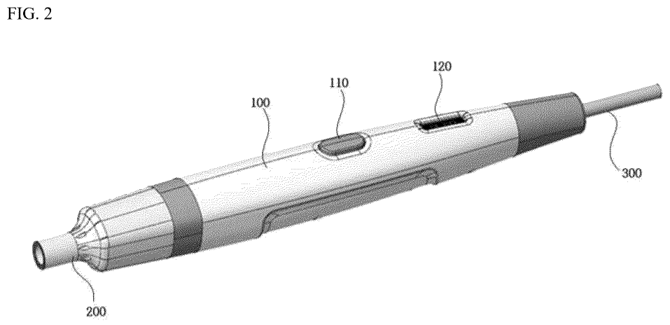

According to an example embodiment of the present disclosure, as shown in FIG. 2, the microneedle therapy device 10 according to an example embodiment of the present disclosure may include an adjustment button 110, and may be configured to start the operation of the microneedles using the motor when the user presses the adjustment button 110. That is, when the user presses the adjustment button 110, operation start information input by the user is transferred to the controller. The controller controls the microneedles to perform one round of operation (that is, a needling operation) in which the microneedles move forward and backward at an initial home position and return to the home position.

According to an example embodiment of the present disclosure, the microneedle therapy device 10 may be set to perform the above-described needling operation each time the user presses the adjustment button 110 (in the manual mode). Further, when the user presses the adjustment button 110, the microneedle therapy device 10 may be set to automatically perform the above-described needling operation (in the automatic mode). Accordingly, when the user sets the automatic mode and presses the adjustment button 110 once, the needling operation of the microneedles may be repeated. This eliminates a need for the user to press the adjustment button 110 each time.

As an example, the operation in the manual mode or the automatic mode may be determined based the input information made by the user. The input information is received by the user interface mounted on the display device 30 of the microneedle therapy device 10. The input information is controlled by the processor of the microneedle therapy device 10 via the cable 300.

Hereinafter, in a case where the microneedle therapy device 10 is set to automatically repeat the needling operation (that is, in the automatic mode), at least one or more of a plurality of settings may be provided to the user such that the user determines how to automatically repeat the needling operation in any manner. Such settings will be described in more detail later.

According to an example embodiment of the present disclosure, in the case where the microneedle therapy device 10 is set such that the needling operation is automatically repeated, the user may set a time interval between needling operations (that is, a first needling operation and a second needling operation).

As an example, the time interval in the automatic mode may be set within a range of about 100 to about 1,000 milliseconds (msec). For example, the time interval may be set to any one of about 100, about 200, about 300, about 400, about 500, about 600, about 700, about 800, about 900, and about 1,000 msec.

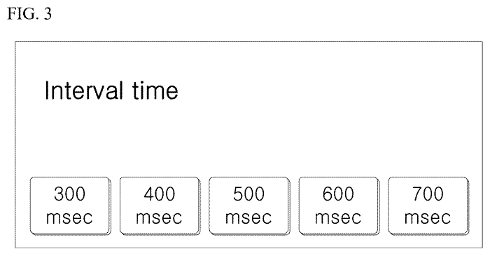

As an example, FIG. 3 is an example of the user interface for receiving the information about the time interval between the needling operations, which is input by the user. The user interface may be provided, for example, when the user selects the automatic mode.

As illustrated in the user interface of FIG. 3, any one of 300, 400, 500, 600, and 700 msec may be selected as the time interval. The user interface shown in FIG. 3 is an example. The user interface of the present disclosure is not limited to the example of FIG. 3. For example, the user may input a desired time interval by typing. Alternatively, upward and downward arrows may be provided as click buttons in the user interface such that the user clicks the click button corresponding to the upward arrow or the downward arrow to increase or decrease the time interval.

According to an example embodiment of the present disclosure, the microneedle therapy device of the present disclosure may be operated in a stacking mode. The “stacking mode” used herein means a mode in which different penetration depths are set for each of a plurality of predetermined consecutive needling operations of the microneedles. Thus, in the stacking mode, the needling operation is consecutively performed twice or more (that is, multiple number of times) in a certain time interval at the same site on the skin. In this case, the time intervals may be set to be identical to or different from each other between the needling operations. As an example, the time interval may be set within a range of approximately 100 to 1,000 msec. For example, the time interval may be set to any one of about 100, about 200, about 300, about 400, about 500, about 600, about 700, about 800, about 900, and about 1,000 msec. The present disclosure is not limited to the above.

The number of times of the needling operations in the stacking mode is not particularly limited as long as it is a plurality of times. For example, the number of times of the needling operations may be two to ten times. The number of times of the needling operations may be appropriately selected by the user depending on a condition of the skin of a person to be treated, or the like. In this case, the needling operation in the stacking mode may be repeatedly performed in the automatic mode.

In another example of the stacking mode, the needling operation may be performed at same or different needle penetration depths. For example, the needle penetration depth may be in a range of approximately more than 0 to 7 mm or less. The needle penetration depth may be selected at an increment of 0.25 mm (for example, 0, 0.25, 0.5, 1 mm, or the like). Further, the needle penetration depth may be selected properly by the user depending on the condition of the skin of the person to be treated, or the like.

In a specific example, the needling operation in the stacking mode is repeatedly performed twice or three times. In this case, the needle penetration depth may be about 0.25 and 6 mm. For example, in the case where the needling operation in the stacking mode is repeatedly performed twice, the needle penetration depth may be 0.25 and 0.5 mm. In the case where the needling operation in the stacking mode is repeatedly performed three times, the needle penetration depth may be 0.25, 0.5 and 0.5 mm, or 0.5, 1 and 1 mm, or the like

For the sake of convenience in description, each needling operation in one stacking mode may be referred to as a “needling stacking” or simply “stacking.” That is, a previous needling operation in one stacking mode may be referred to as a “first (needling) stacking,” and a subsequent needling operation may be referred to as a “second (needling) stacking.” In some cases, a first needling operation may be referred to as a “first (needling) stacking”, and a second needling operation may be referred to as a “second (needling) stacking.”

Further, for example, in one stacking mode in which the stacking is repeated three times, such automatically-repeated three stackings are referred to as a “stacking set.” That is, a first round of three repetitive stackings is referred to as a “first stacking set,” and a second round of three repetitive stackings is referred to as a “second stacking set.”

As described above, the needle penetration depth may be differently set for each (needling) stacking. The needle penetration depth in each (needling) stacking may be set to the same as that in a previous (needling) stacking, and may be set to be different from that of the previous (needling) stacking.

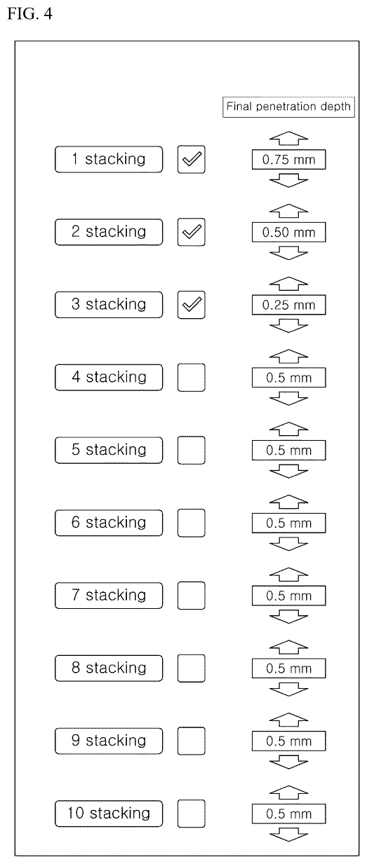

Referring to FIG. 4, the stacking mode is set such that three (needling) stackings are performed. The first stacking is set such that the needling operation is performed at the needle penetration depth of 0.75 mm. The user may increase or decrease the needle penetration depth by clicking the upward arrow or the downward arrow. That is, in the first stacking, the microneedle moves forward by the needle penetration depth of 0.75 mm from the home position and then moves backward to return to the home position. The second stacking is set such that the needling operation is performed at the needle penetration depth of 0.50 mm. When the time interval is set to, for example, 100 msec, the second stacking is performed after 100 msec during which the first stacking is performed. That is, the microneedle, which has been returned to the home position, moves forward by the needle penetration depth of 0.50 mm and then moves backward to return to the home position. Similarly, a third stacking is performed after 100 msec during which the second stacking is performed. The microneedle, which has been returned to the home position, moves forward by the needle penetration depth of 0.25 mm and then moves backward to return to the home position. Such a stacking mode is repeatedly performed. In other words, after the first stacking set in which the above-described stacking is repeated three times, that is, after 100 msec, a first stacking of the second stacking set is performed again. In the example above, the first stacking and the second stacking, and the second stacking and the third stacking have been described to be performed at the time interval of 100 msec. Alternatively, these stacking may be performed at different time intervals. For example, the first stacking and the second stacking may be performed at the time interval of 100 msec, and the second stacking and the third stacking may be performed at the time interval of 200 msec.

In the stacking mode described above, the user presses the adjustment button 110 once such that the microneedle penetrates through the same site on the skin several times. Thus, by the mechanism of the microneedling and the injection of the effective ingredients (pharmacological agent or the like), it is possible to treat several layers of the skin (for example, a papillary layer, a plexiform layer and the like) at once. This is, it is possible to treat the entire layer of the skin. The term “skin” used here should be understood to include the scalp as a whole skin. For example, the microneedle therapy device of the present disclosure may be used to inject the effective ingredients into the scalp.

For example, in a case of a normal mode other than the stacking mode, when the user wants to perform 100 needling operations at a first penetration depth and a second penetration depth, which are different from each other, with respect to the entire skin of the face, the needle penetration depth of the microneedle therapy device needs to be set to the first penetration depth such that the microneedle therapy device moves on the entire skin of the face to perform 100 needling operations, and subsequently, the needle penetration depth of the microneedle therapy device needs to be set to the second penetration depth such that the microneedle therapy device moves along the same path again on the entire skin of the face to performed 100 needling operations (which is referred to as a “Pass mode”). On the other hand, in the case of the stacking mode, the first stacking is set to be performed at the first penetration depth and the second stacking is set to be performed at the second penetration depth. Next, the microneedle therapy device moves on the entire skin of the face once to perform 200 needling operations (that is, the stacking mode is repeated 100 times). As described above, the microneedle therapy device may treat several layers of the skin at once without repeatedly moving several times along the same path on the entire skin of the face

In an example embodiment, the stacking mode of the present disclosure may be performed in either the manual mode or the automatic mode according to a selection made by the user. The manual mode means that the user input information is required every time the first stacking set performed at a first skin position proceeds to the second stacking set to be performed at a second skin position. The automatic mode means that the first stacking set automatically proceeds to the second stacking set based on a first round of user input information. In this case, a plurality of needling stackings in the stacking mode may be performed in the automatic mode regardless of whether the stacking mode is set to the manual mode or the automatic mode.

Detailed functions of the above-described stacking mode, that is, the number of times of the needle stackings, the time interval, the needle penetration depth, the selection of the manual mode or the automatic mode between the stacking sets, and the like, may be determined based on the user input information.

The term the “user input information” used here may be information or signal transferred when the user merely presses the adjustment button 110 or a similar component, or may be information or signal transferred when the user selects specific relevant information provided on the user interface (for example, the interfaces illustrated in FIGS. 3 to 5).

The microneedle therapy device of the present disclosure is not limited to the configuration which generates the positive pressure. Alternatively, the microneedle therapy device of the present disclosure may be configured such that the user sets a positive-pressure generation time point in the stacking mode in addition to the generation of the positive pressure.

Hereinafter, adjusting the positive-pressure generation time point during the needling operation and setting the positive-pressure generation time point in the stacking mode will be sequentially described.

### 1) Adjusting Positive-Pressure Generation Time Point During Needling Operation

According to an example embodiment of the present disclosure, a positive pressure may be generated by a pressure generation mechanism and a (solenoid) valve provided in the microneedle therapy device 10, and a positive-pressure generation time point may be adjusted by controlling an operation of the pressure generation mechanism and a time point at which the (solenoid) valve is open or closed. Accordingly, in consideration of a condition of the skin to be treated, it is possible to optimize a profile in which the effective ingredients are injected into the skin (a profile in which the effective ingredients are injected to correspond to different depths of the skin). That is, by differently setting the positive-pressure generation time point during the needling even if the final penetration depths of the needling are the same, it is possible to differently setting the profile in which the effective ingredients are injected into the skin.

For example, the effective ingredients may be injected uniformly regardless of the different depths of the skin. Alternatively, the effective ingredients may be injected intensively at a specific depth. Such an action may be further maximized by adjusting power of the pneumatic pump 150.

That is, by generating and maintaining a predetermined positive pressure as soon as the microneedles begin to penetrate the skin (or before the penetration), it is possible to uniformly inject the effective ingredients regardless of the different depths of the skin. Alternatively, when the microneedle reaches the specific depth, the compressed air of a relatively high pressure may be blown into the microneedles to intensively inject the effective ingredients to the specific depth. In the case, the specific depth may be different from that at which the microneedles will finally penetrate the skin (hereinafter referred to as “final penetration depth”).

The user may input, as pieces of setting information, a desired positive-pressure generation time (the needle penetration depth of the microneedles at the time of applying the positive pressure, that is, the specific depth) and a desired power of the pneumatic pump 150, with the user interface of the display device 30. The pieces of setting information are transmitted to a processor of the microneedle therapy device 10. The processor controls the opening/closing of the (solenoid) valve and the power of the pressure generation mechanism based on the pieces of setting information.

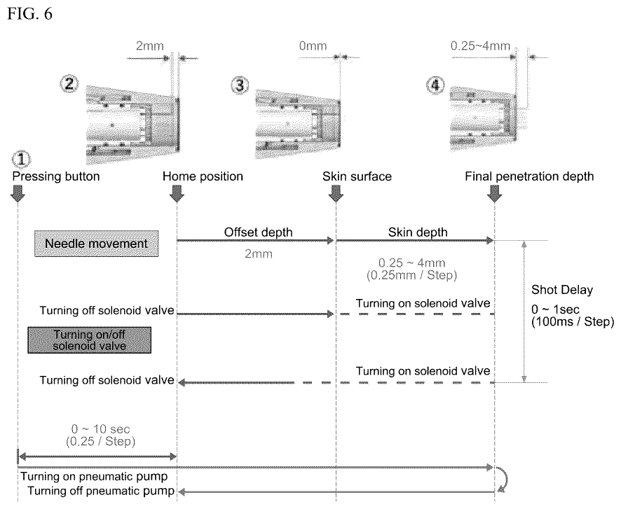

FIG. 6 illustrates an operation of the pressure generation mechanism (for example, the pneumatic pump) and a sequence of controlling the opening/closing of the solenoid valve according to an example embodiment of the present disclosure.

Hereinafter, a manual mode is premised and an automatic mode is understood to automatically execute a sequence of the manual mode to be described.

When the adjustment button 110 is pressed by the user, the microneedle therapy device 10 starts to operate.

In an initial state of the microneedle therapy device 10 before the user presses the adjustment button 110, the pneumatic pump remains turned off and the solenoid valve also remains turned off (the solenoid valve remains closed). When the user presses the adjustment button 110, the pneumatic pump is turned on to generate the compressed air. The flow of the compressed air is blocked by the solenoid valve. The microneedles are in the home position state (for example, the microneedles are offset by 2 mm inward from the tip end of the needle cover).

When the adjustment button 110 is pressed by the user and a predetermined time period (about 10 seconds or less) elapses, the microneedle starts to move forward. When the microneedles are moved by an offset distance, the microneedles come into contact with the skin, and subsequently penetrate the skin. In the case, the offset distance may be set in a range of more than zero to 7 mm or less according to a configuration of the microneedle unit and user's needs. For example, the offset distance may be set in increments of 0.25 mm in the range of more than zero to 7 mm or less. As an example, when the offset distance is set in the range of more than zero to 1 mm or less, it may be set to 0.25 mm, 0.5 mm, 0.75 mm, and 1 mm. Even in the remaining range of more than 1 mm to 7 mm or less, the offset distance may be set in the same manner as the above.

In the present disclosure, the positive pressure is generated by turning (opening) a solenoid valve 154. That is, a time point at which the solenoid valve 154 is turned on may be the positive-pressure generation time point.

The profile in which the effective ingredients are injected to correspond to different depths of the skin may vary depending on the positive-pressure generation time point, a magnitude of the positive pressure, a type of effective ingredients (that is, skin booster) or the like. Further, a skin state at a site to be treated (for example, a position or state of the epidermal layer, a position or state of the dermal layer, or the like) varies depending on a person to be treated and a skin site of the person (forehead site, eye site or the like).

In the present disclosure, the user may set the positive-pressure generation time point or the magnitude of the positive pressure in consideration of the type of skin booster, a skin condition of a site to be treated, and the like to obtain a profile in which desired effective ingredients are injected.

For example, when wanting effective ingredients to be intensively injected at a specific depth of 3 mm in consideration of a position of the epidermal layer or a position of the dermal layer at the skin site to be treated, the user may set the positive-pressure generation time point or the magnitude of the positive pressure corresponding thereto. The specific depth of 3 mm mentioned as above is merely an example. The specific depth may be set in a range of more than zero to 7 mm or less. For example, when the specific depth is set in increments of approximately 0.25 mm in the range of approximately more than zero to 1 mm or less, the specific depth may be set to approximately 0.25 mm, 0.5 mm, 0.75 mm, and 1 mm. Even in the remaining range of approximately more than 1 mm to 7 mm or less, the specific depth may be set in the same manner as the above.

According to an example embodiment of the present disclosure, the positive-pressure generation time point may be set to correspond to a penetration depth of the microneedles at the positive-pressure generation time point so that the user may intuitively set the positive-pressure generation time point. For example, in a case where the user sets to a depth of 3 mm as the positive-pressure generation time point (the needle penetration depth of the microneedles at the positive-pressure generation time point), when the needle penetration depth of the microneedles is 3 mm, the solenoid valve is open to generate the positive pressure.

Further, according to the profile in which the desired effective ingredients are injected, the positive-pressure generation time point may be set while the microneedles move forward, or may be set while the microneedles move backward.

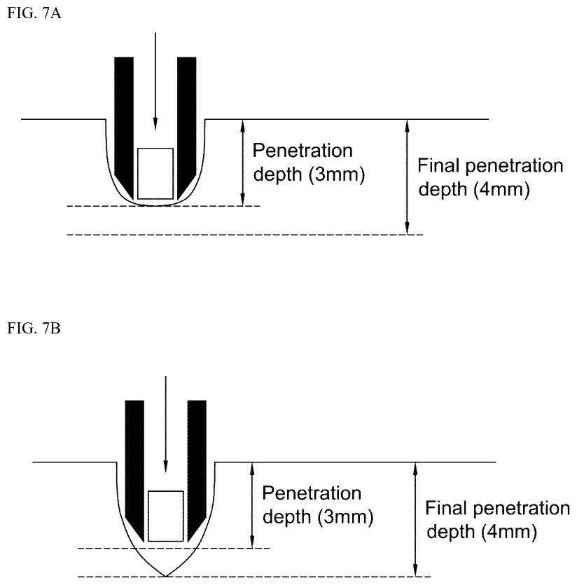

For example, FIG. 7A illustrates a state in which, when the final penetration depth is 4 mm, the microneedle moves forward by 3 mm. FIG. 7B illustrates a state in which, when the final penetration depth is 4 mm, the microneedle penetrates by 4 mm, which is the final penetration depth, and subsequently move backward by 1 mm. FIGS. 7A and 7B illustrate states in which, when the needle penetration depth of the microneedle is 3 mm, shapes formed in the skin by the penetration of the microneedle are different from each other. In this case, the profile in which the effective ingredients are injected may vary.

For example, as illustrated in FIG. 7B, in the case where the positive pressure is generated while the microneedle penetrates by the final penetration depth of 4 mm and subsequently moves backward by 1 mm, the effective ingredients may be easily injected to a site from which the microneedle is just withdrawn. In particular, the effective ingredients may be intensively injected around the depth of 3 mm.

In addition, the positive pressure may be maintained for a predetermined period of time even after the microneedle has completely withdrawn from the skin. With this configuration, even after the microneedle has completely withdrawn from the skin, the effective ingredients may be more easily injected to the site in the epidermal layer from which the microneedle is withdrawn.

### 2) Setting Positive-Pressure Generation Time Point in Stacking Mode

As described above, the stacking mode is a mode in which different penetration depths are set for each of a plurality of predetermined consecutive needling operations of the microneedle in one operation mode. The stacking mode may be repeatedly performed.

According to an example embodiment of the present disclosure, the user may set the positive-pressure generation time point for each needling operation in one stacking mode. For example, in a case where the needling stacking in which one stacking mode is repeated twice is performed, the positive-pressure generation time point in the first needling stacking and the positive-pressure generation time point in the second needling stacking (for example, the needle penetration depth of the microneedle at the positive-pressure generation time point, that is, a target depth) may be identical to or different from each other. These positive-pressure generation time points may be determined according to the user input information.

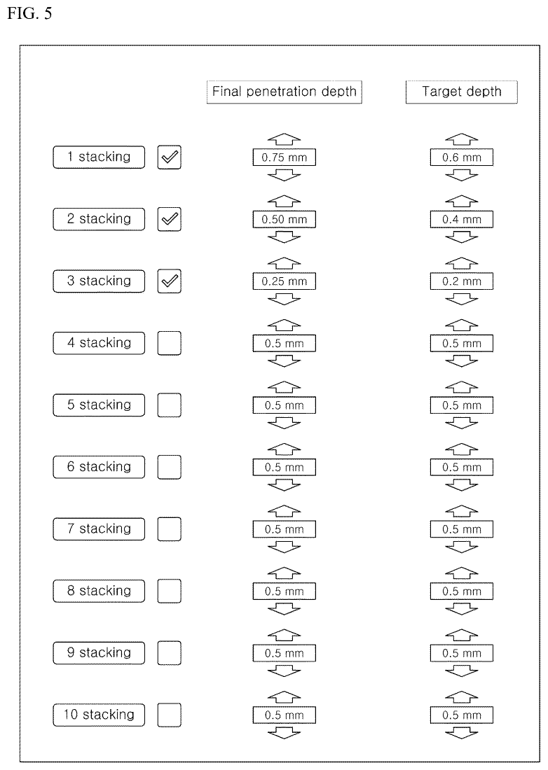

For example, FIG. 5 is another example of the user interface for setting the stacking mode. The user interface may be provided when the user clicks a time interval button on the user interface illustrated in FIG. 4.

As shown in FIG. 5, in the user interface for setting the stacking mode, in addition to the setting of the number of needling operations in the stacking mode and the needle penetration depth for each needling operation, the positive-pressure generation time point for each needling operation may be set.

Referring to FIG. 5, the stacking mode is set such that three (needling) stackings are performed. The first stacking is set such that the needling operation is performed at the needle penetration depth of 0.75 mm and the positive-pressure generation time point is set at the target depth of 0.6 mm. The second stacking is set such that the needling operation is performed at the needle penetration depth of 0.5 mm and the positive-pressure generation time point is set at the target depth of 0.4 mm. The third stacking is set such that the needling operation is performed at the needle penetration depth of 0.25 mm and the positive-pressure generation time point is set at the target depth of 0.2 mm.

With this configuration, the user may set a final penetration length of the microneedle and the positive-pressure generation time point (the penetration depth of the microneedle when the positive-pressure is generated, that is, the target depth) for each stacking in the stacking mode, thereby optimizing a profile in which the effective ingredients are injected to the skin for each stacking. In other words, it is possible not only to treat several layers of the skin at once, but also to optimize the profile in which the effective ingredients are injected to each layer of the skin to be treated.

The microneedle therapy device, specifically the positive-pressure generation apparatus, which operates in the stacking mode of the present disclosure, is configured to repeatedly insert the microneedle into the skin by a precise depth. Accordingly, when a product such as a pharmacological agent, specifically a skin booster is used together, the pharmacological agent applied to the skin may be easily delivered to a desired site. Further, pneumatic gas may be repeatedly sprayed to the fibrous beneath the skin (air pockets are generated). This makes it possible to achieve an improvement in skin texture, a collagen promotion, or the like due to the pharmacological agent and easily achieve the so-called “sub-cision” effect by breaking the fiber throughout the scar of the skin.

A configuration of the microneedle therapy device according to an example embodiment of the present disclosure will be described below.

In an example embodiment of the present disclosure, the microneedle therapy device includes:
needle cover;
a pressure generation mechanism, for example, a pneumatic pump; or a gas tank including a gas injectable to the skin, such as a CO₂ gas, an oxygen gas, a normal atmospheric gas, or the like (including a case where a gas regulator is provided),
a valve configured to be open and closed to supply a compressed air or compressed gas (also collectively referred to as a “compressed gas”) from the pressure generation mechanism into the needle cover; and
a microneedle disposed inside the needle cover and configured to move forward and backward via an opening of the needle cover,
wherein, the processor controls the opening and closing of the valve and the movement of the microneedle, and
wherein an open time point of the valve may be controlled based on the user input information.

In this case, the microneedle may have either a hollow structure or a solid structure, and may have a size of 15 gauge (G) to 33 G. For example, in the hollow structure, the size may be 28, 29, 30, 31 or 32 G. In the solid structure, the size may be 28, 29, 30, or 31 G.

In an example embodiment of the microneedle therapy device, a case where the pneumatic pump is used as the pressure generation mechanism will be described later.

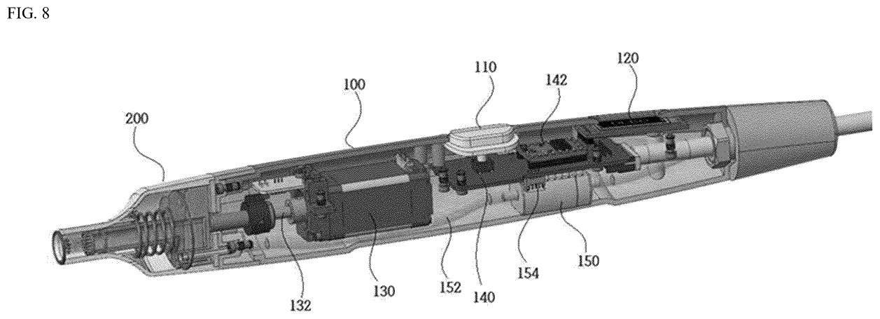

As an example, as illustrated in FIGS. 2 and 8, the microneedle therapy device 10 according to the present disclosure includes a body housing 100.

The body housing 100 defines an appearance of the microneedle therapy device 10 according to the present disclosure. ‘In this case, a microneedle unit 200 is assembled to a front end of the body housing 100. The cable 300 is connected to a rear end of the body housing 100. The adjustment button 110 is provided in one portion of the body housing 100. A ventilation port 120 is provided in another portion of the body housing 100. Various functions such as power On-Off, the start of operation, mode switching, power adjustment, and the like may be set or adjusted by the adjustment button 110.

Further, the ventilation port 120 is a passage through which external air may be introduced into the body housing 100. A filter may be used as the ventilation port 120 if necessary.

Further, a drive power source 130 is provided inside the body housing 100. Preferably, a known linear motor may be used as the drive power source 130. In this case, unlike a typical electric motor in which a rotor rotates inside a stator, the linear motor is configured so that a mover moves linearly relative to a deployed stator at a gap along an extension line of the stator. By using the linear motor configured as above, it is possible to provide a structure in which a linear reciprocating motion rather than a rotational motion is implemented in a narrow space. A movable rod 132, which corresponds to the mover, may be provided in the drive power source 130 to be protruded therefrom. The movable rod 132 reciprocates linearly within a certain distance.

Further, a main board 140 is provided inside the body housing 100 to be spaced apart from the drive power source 130. The controller 142 is mounted on the main board 140.

In this case, the main board 140 as a printed circuit board (PCB) is a kind of microcomputer which is connected to the adjustment button 110 to provide various functions for the operation, control, setting and the like of the microneedle therapy device according to the present disclosure based on a control signal from the controller 142. To do this, the drive power source 130 is also controlled by the controller 142.

Further, a pneumatic pump 150 is provided below the main board 140 to be spaced apart from the drive power source 130. The pneumatic pump 150 is configured to compress air at a constant pressure and supply the same to the microneedle unit 200 assembled to the front end of the body housing 100. To do this, a pneumatic hose 152 is connected between the pneumatic pump 150 and the microneedle unit 200. In particular, a solenoid valve 154 may be provided in the pneumatic pump 150 to supply a certain amount of compressed air only at the time of need. In this case, the solenoid valve 154 is electrically connected to the controller 142 and an operation thereof is controlled based on a control signal from the controller 142.

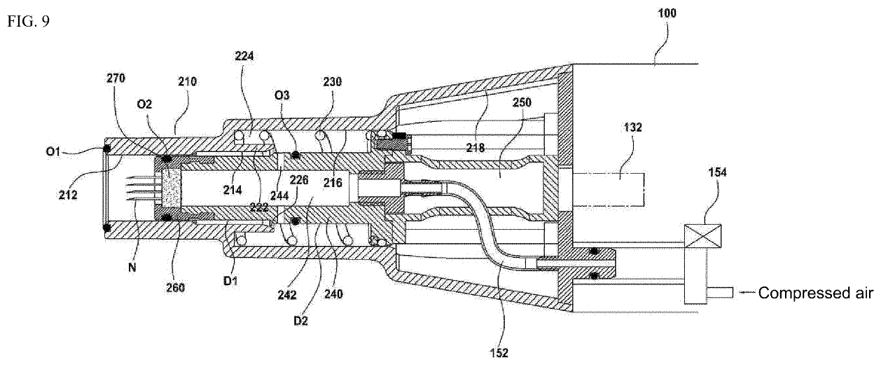

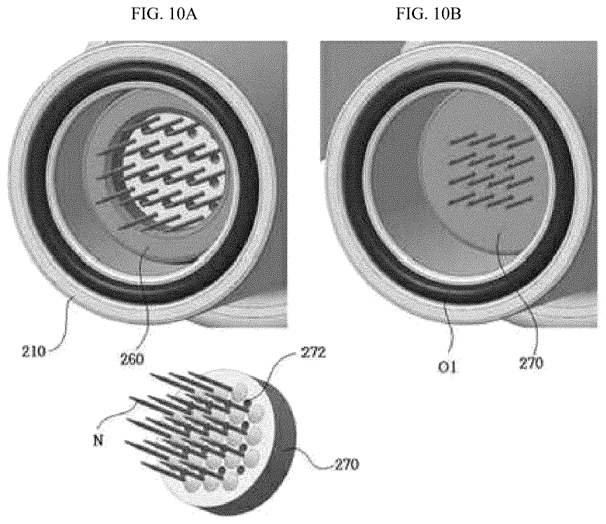

Further, as illustrated in FIG. 9 and FIG. 10, the microneedle unit 200 includes a needle cover 210. The needle cover 210 is assembled to the front end the body housing 100. The needle cover 210 may be formed to have a stepped structure in which a first inner-diameter portion 212, a second inner-diameter portion 214, a third inner-diameter portion 216 and a fourth inner-diameter portion 218 are sequentially formed from the front end to the rear end of the needle cover 210.

In this case, the fourth inner-diameter portion 218 assembled to the body housing 100 has the largest inner diameter, and the third inner-diameter portion 216, the second inner-diameter portion 214, and the first inner-diameter portion 212 is sequentially reduced in inner diameter. In particular, one side of the second inner-diameter portion 214 protrudes to form an inner boss 222 so that a certain size of locking groove 224 is formed between an outer circumferential surface of the inner boss 222 and the third inner-diameter portion 216.

In this case, the locking groove 224 has a substantially U shape in a cross-sectional view. An end portion of the inner boss 222, that is, an end portion of the second inner-diameter portion 214, is chamfered inward to form a tapered surface 226. The tapered surface 226 is used to increase airtightness by a third O-ring O3 (to be described later).

Further, a coil spring 230 is inserted into the third inner-diameter portion 216. A front end of the coil spring 230 is locked to the locking groove 224. In addition, a rear end of the coil spring 230 is locked to the plunger 240. Therefore, the plunger 240 is configured to be elastically pushed by the coil spring 230.

The plunger 240 is formed as a cylindrical member having an inner hollow portion 242. A rear end of the plunger 240 is flange-fixed to one end of a connection tube 250. In addition, a rear end of the connection tube 250 is connected and fixed to a front end of the movable rod 132. Therefore, when the movable rod 132 reciprocates forward and backward, the connection tube 250 also moves so that the plunger 240 reciprocates forward and backward.

In addition, the pneumatic hose 152 is provided to penetrate the connection tube 250. An end portion of the pneumatic hose 152 is connected and fixed to the hollow portion 242 of the plunger 240 such that they are in communication with each other. In this case, the pneumatic hose 152 provided to penetrate the connection tube 250 is configured to be flexible. Thus, when the connection tube 250 moves, the pneumatic hose 152 smoothly guides the motion of the connection tube 250 without being cut. Of course, the motion range of the connection tube 250 is small, so it's not matter.

In addition, an exhaust hole 244 is formed in a periphery of the plunger 240. The exhaust hole 244 is in communication with the hollow portion 242. Specifically, the exhaust hole 244 is formed outward to be spaced apart from the end portion of the inner boss 222 when the plunger 240 is located at a home position. That is, the plunger 240 has a first outer-diameter portion D 1 corresponding to the first inner-diameter portion 212 and a second outer-diameter portion D2 corresponding to the second inner-diameter portion 214 which are provided sequentially from the front end to the rear end of the plunger 240. The exhaust hole 244 is formed in the second outer-diameter portion D2. A position immediately before the second outer-diameter portion D2 is inserted into the second inner-diameter portion 214 is the home position.

Therefore, at the home position, the exhaust hole 244 is positioned to be spaced apart from the tapered surface 226 without being inserted into the second inner-diameter portion 214.

Further, the third O-ring O3 is fixed to the second outer-diameter portion D2 to be spaced apart from the exhaust hole 244.

The third O-ring O3 may be fitted into and fixed to an O-ring groove (not illustrated) formed in the second outer-diameter portion D2, and may be formed to be spaced apart from the exhaust hole 244 toward the connection tube 250.

Further, as illustrated in FIGS. 9 and 10A, a cylindrical fixture 260 is fastened to the front end of the plunger 240.

In this case, a sealing plate 270 is provided inward of the fixture 260 to hermetically seal an opening of the frond end of the plunger 240.

To this end, a stepped portion may be formed in an inner circumferential surface of a front end of the fixture 260. Thus, the sealing plate 270 may be tightly locked to the stepped portion so that the sealing plate 270 may be firmly assembled and fixed to the front end of the plunger 240.

In addition, a plurality of microneedles N is fixed to the sealing plate 270. Each microneedle N has an inner hollow. A rear end of the microneedle N passes through the sealing plate 270 to be in communication with the hollow portion 242. With this configuration, air may flow into the first inner-diameter portion 212 via a sequence of the hollow portion 242 and the microneedles N.

Further, a second O-ring O2 is provided in an outer circumferential surface of the front end of the fixture 260 to maintain airtightness between the first inner-diameter portion 212 and the fixture 260. Further, a first O-ring O1 may be embedded in a front end surface of the needle cover 210 to maintain airtightness between the front end surface and the skin.

Further, a plurality of discharge holes 272 may be formed in the sealing plate 270. The discharge holes 272 may be formed to penetrate the sealing plate 270 such that they are in communication with the hollow portion 242. The discharge holes 272 may be provided to flow the air therethrough. That is, in this case, the microneedle may have a solid structure and the air may flow through the discharge holes 272.

Alternatively, as illustrated in FIG. 10B, the sealing plate 270 may be directly fixed to the front end of the plunger 240 without the fixture 260. In this case, the discharge holes 272 may be omitted and the air may flow only through the microneedles N. For example, as illustrated in FIG. 10B, the microneedle may be configured to have a hollow structure.

An operation of the microneedle therapy device 10 configured as above according to the present disclosure and a method for using the microneedle therapy device 10 will be described later.

First, prior to the description of the operation of the microneedle therapy device 10 and the method for using the microneedle therapy device 10, the microneedle therapy device 10 according to the present disclosure will be conceptually described with a focus on obvious differences from a technology in the related art.

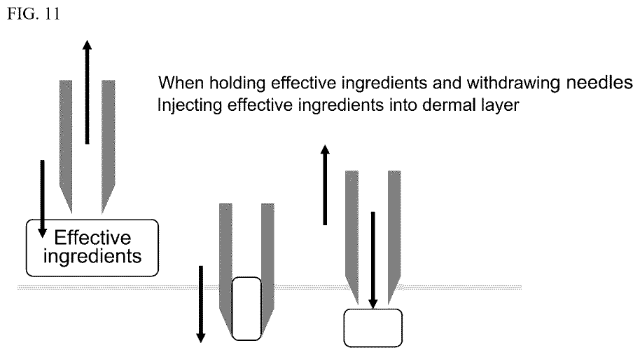

As illustrated in FIG. 11, the microneedle therapy device 10 according to an example embodiment of the present disclosure uses a method for applying effective ingredients such as pharmacological agents to the skin and injecting the same into a dermal layer of the skin with the microneedles N instead of using an injection liquid. FIG. 11 illustrates an example embodiment in which hollow microneedles N are used.

As described above, the microneedle therapy device 10 according to an example embodiment of the present disclosure does not use the injection liquid. Therefore, the present disclosure is not regulated by medical treatment laws. Thus, the present disclosure may be applied to various skin boosters other than limited effective ingredients.

That is, as illustrated in FIG. 11, effective ingredients may be injected to the skin by applying a skin booster as effective ingredients to the skin, followed by bring each microneedle N close to the skin so that the effective ingredients is received in the central hollow of each microneedle N, followed by penetrating each microneedle N into the skin, followed by blowing compressed air into the central hollow of each microneedle N at the time of penetrating each microneedle N into the skin or after each microneedle N penetrates into the skin, followed by withdrawing each microneedle N from the skin.

In addition, when the microneedle therapy device of the present disclosure is used, air pockets may be generated beneath the skin. In particular, when the microneedle therapy device is used with respect to a skin layer with many wrinkles or scars, fiber bands adhered to bottoms of the wrinkles or scars may be cut to promote collagen synthesis and create new blood vessels. This provides the effect of alleviating wrinkles or scars.

Next, the operation of the microneedle therapy device 10 according to an example embodiment of the present disclosure and the method for using the same will be described in detail.

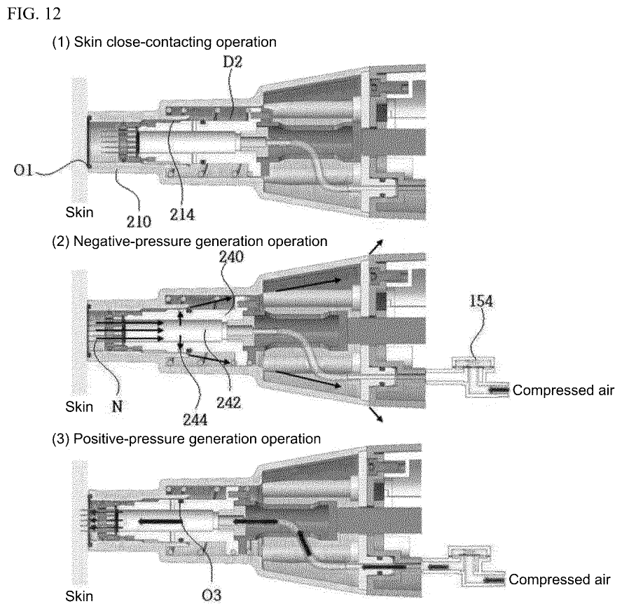

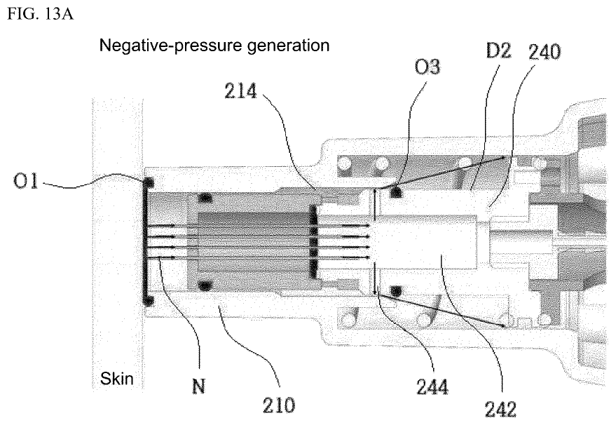

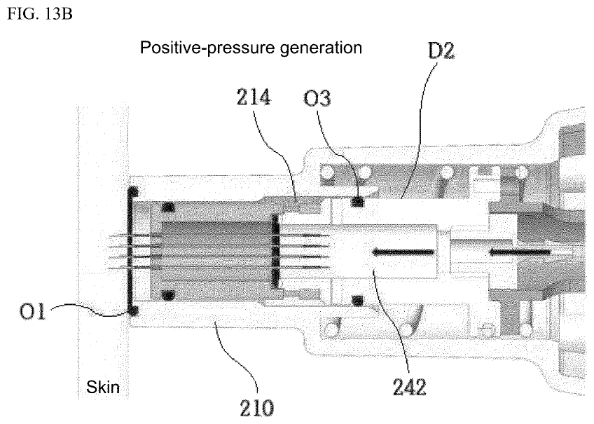

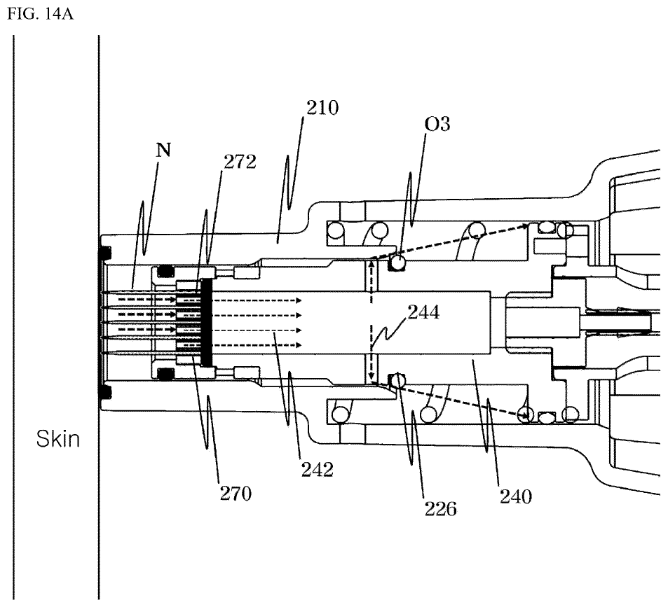

As illustrated in FIG. 12, the method for using the microneedle therapy device 10 according to an example embodiment of the present disclosure may include a skin close-contacting operation, a negative-pressure generating operation and a positive-pressure generating operation. Hereinafter, the method for using the microneedle therapy device 10 illustrated in FIG. 12 will be described with reference to an example embodiment of the microneedles N of a hollow type illustrated in FIG. 13.

First, the skin close-contacting operation is an operation of bringing the front end of the needle cover 210 of the microneedle unit 200 into close contact with the skin in a state in which effective ingredients are applied onto the skin, and forming a closed space inside the needle cover 210.

According to an example embodiment of the present disclosure, the first O-ring O1 provided in the front end surface of the needle cover 210 is brought into close contact with the skin to form the closed space. In this case, the microneedles N are in a home position state. The home position state means a state in which that the microneedles N are arranged to be spaced apart from the front end of the needle cover 210, and just before the second outer-diameter portion D2 is inserted inward of the second inner-diameter portion 214.

In the home position state, the pneumatic pump 150 is operated to generate the compressed air, but the supply of the compressed air is blocked by the solenoid valve 154.

The negative-pressure generating operation is an operation of discharging, inward of the thirst inner-diameter portion 216, air existing in the closed space between the skin and the front end of the plunger 240 via the exhaust hole 244 of the plunger 240 and the tapered surface 226 of the inner boss 222 while moving the plunger 240 forward, and forming a negative pressure in the closed space.

In this case, the air in the closed space moves to the hollow portion 242 of the plunger 240 via the inner hollow of each microneedle N and is discharged to the outside via the exhaust hole 244 before the tapered surface 226 is sealed by the third O-ring O3.

Further, the plunger 240 is moved with the operation of the drive power source 130, which is a linear motor.

As described above, when the negative pressure is generated in the closed space, the effective ingredients applied onto the skin may be easily flown into the inner hollow of each microneedle N. At this time, the supply of the compressed air remains blocked by the solenoid valve 154.

The positive-pressure generating operation is an operation of further moving the plunger 240 forward such that the microneedles N penetrate the skin while holding the effective ingredients, hermetically sealing the tapered surface 226 with the third O-ring 03 and simultaneously opening the solenoid valve 154 to supply the compressed air such that the effective ingredients are injected into the skin, and subsequently, moving the plunger 240 backward and withdrawing the microneedles N from the skin to further supply the compressed air for several seconds such that the closed space is kept at a positive pressure.

A sequence of operations as described above may provide the same effects as those of directly injecting effective ingredients into the skin with needles. This makes it possible to inject the effective ingredients into the skin in a more smooth and efficient manner.

In this case, the coil spring 230 may perform a function of applying an elastic force so that the plunger 240 may smoothly return to the home position.

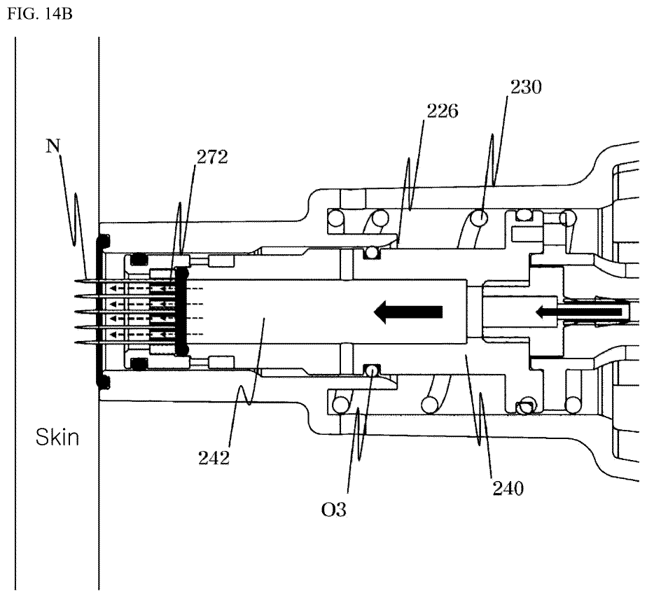

Alternatively, the microneedles N of the present disclosure may have a solid structure as illustrated in FIGS. 14A and 14B, instead of the hollow structure described above with reference to FIGS. 13A and 13B.

For example, as illustrated in the figure, the microneedles N may be configured to have a solid structure whose interior is filled. The sealing plate 270 may have a structure in which the plurality of discharge holes 272 is formed between the microneedles N penetrating through the sealing plate 270.

The microneedles N having such a solid structure may be operated as illustrated in FIGS. 14A and 14B. The operation of the microneedles N having the solid structure is substantially similar to that of the microneedles N having the hollow structure as described above, and therefore, only differences between the operation of the microneedles N having the solid structure and the operation of the microneedles N having the hollow structure will be additionally described below.

That is, when the front end of the needle cover 210 is brought into close contact with the skin in the state in which the effective ingredients are applied onto the skin, the closed space is formed between the skin and the needle cover 2100, and the air existing in the closed space moves to the inner hollow portion 242 of the plunger 240 and is discharged along the tapered surface 226. As a result, the closed space is kept at the negative pressure as illustrated in FIG. 14A.

In this state, as illustrated in FIG. 14B, the plunger 240 is moved forward and the microneedles N penetrates the skin. When the third O-ring O3 is brought into close contact with the tapered surface 226 and the compressed air is supplied to the inner hollow portion 242 of the plunger 240, the closed space is kept at the positive pressure so that the effective ingredients applied on the skin are injected into the skin. The effective ingredients may be injected via gaps between outer circumferential surfaces of the microneedles N and the skin. Thereafter, by moving the plunger 240 backward, further supplying the compressed air for several seconds while withdrawing the microneedles N, and keeping the closed space remains at the positive pressure, the effective ingredients may be smoothly and efficiently injected and filled into sites from which the microneedles N are withdrawn.

A sequence of operations as described above may provide the same effects as those of directly injecting effective ingredients into the skin with needles. This makes it possible to inject the effective ingredients into the skin in a more smooth and efficient manner.

In this case, the coil spring 230 may perform a function of applying an elastic force so that the plunger 240 may smoothly return to the home position.

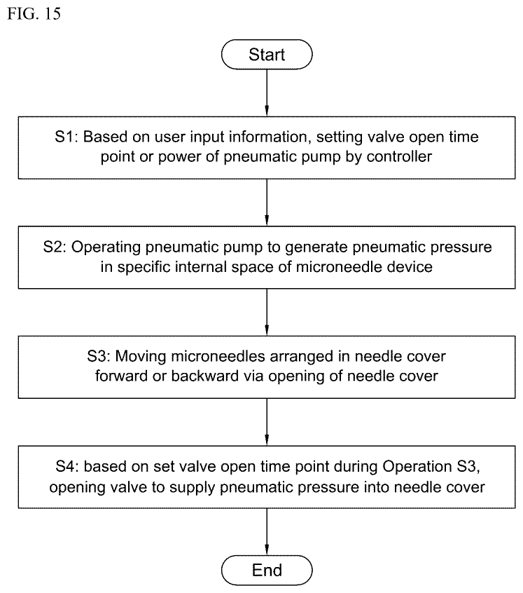

FIG. 15 is a flowchart exemplarily explaining a method for injecting effective ingredients into the skin using the microneedle therapy device according to an example embodiment of the present disclosure.

As illustrated in FIG. 15, based on information input by the user (which is transmitted to the controller), the controller may set the valve opening time point or the power of the pneumatic pump (Operation S1). Subsequently, when the user presses the adjustment button, the pneumatic pump is operated to generate a pneumatic pressure in a specific internal space of the microneedle therapy device (Operation S2). The microneedles N arranged in the needle cover move forward or backward via the opening of the needle cover (Operation S3). During the operation of Operation S3, the solenoid valve provided between the inner space and the needle cover is open based on the set valve opening time point to supply the pneumatic pressure generated by the pneumatic pump into the needle cover (Operation S4).

### EXPLANATION OF REFERENCE NUMERALS

1: Micro needle therapy system
10: Microneedle therapy device
30: Display device
100: Main housing
110: Adjustment button
130: Driving source
140: Main board
142: Controller
150: Pneumatic pump
152: Pneumatic hose
154: Solenoid valve
200: Microneedle unit
230: Coil spring
240: Plunger
250: Connection tube
260: Fixture
270: Sealing plate
300: Cable

What is claimed is:

1. A microneedle therapy device comprising:
a memory storing one or more instructions; and
at least one processor configured to execute the one or more instructions stored in the memory to control a needling in which a microneedle moves forward and backward at a home position and returns to the home position,
wherein the at least one processor is configured to execute the one or more instructions to receive user input information about the microneedle therapy device, which is input by a user, and performs a stacking mode based on the user input information, and
wherein the stacking mode repeatedly performs a stacking set in which a needling stacking is consecutively performed two or more times at a same or different time intervals at a same position on a skin.

2. The microneedle therapy device of claim 1, wherein the needling stacking in the stacking mode is repeatedly performed twice to 10 times.

3. The microneedle therapy device of claim 1, wherein the needling stacking in the stacking mode is performed at needle penetration depths which are identical to or different from each other.

4. The microneedle therapy device of claim 3, wherein each of the needle penetration depths is in a range of more than 0 to 7 millimeter (mm) or less.

5. The microneedle therapy device of claim 1, wherein the needling stacking in the stacking mode is repeatedly performed twice or three times, and
wherein needle penetration depths are identical to or different from each other in a range of 0.25 to 6 mm.

6. The microneedle therapy device of claim 1, wherein the stacking mode is performed in a manual mode or an automatic mode,
wherein, in the manual mode, the user input information is required each time when a first stacking set performed at a first skin position proceeds to a second stacking set performed at a second skin position, and
wherein, in the automatic mode, the first stacking set automatically proceeds to the second stacking set based on a first round of user input information initially input by the user.

7. The microneedle therapy device of claim 1, wherein the microneedle has a hollow or solid structure.

8. The microneedle therapy device of claim 1, further comprising:
a needle cover;
a pressure generation mechanism;
a valve configured to be open and closed to supply a compressed gas from the pressure generation mechanism inward of the needle cover; and
a microneedle arranged inside the needle cover and configured to move forward or backward via an opening provided in the needle cover,
wherein the at least one processor controls the opening and closing of the valve and the movement of the microneedle, and controls a valve open time point at which the valve is opened based on the user input information.

9. The microneedle therapy device of claim 8, wherein the pressure generation mechanism is configured to generate the compressed gas and supply the compressed gas inward of the needle cover.

10. The microneedle therapy device of claim 8, wherein the at least one processor controls a power of the pressure generation mechanism based on the user input information.

11. The microneedle therapy device of claim 8, wherein the valve is configured to be open while the microneedle penetrates the skin.

12. The microneedle therapy device of claim 8, wherein the valve is configured to be open while the microneedle is withdrawn from the skin after reaching a final penetration depth.

13. The microneedle therapy device of claim 8, wherein the valve is configured to be open for a predetermined period of time after the microneedle is completely withdrawn from a skin.

14. The microneedle therapy device of claim 8, wherein the user input information is information about a penetration depth at which the microneedle penetrates the skin at the valve open time point that the user wants.

15. A microneedle therapy system, comprising:
the microneedle therapy device of claim 1; and
a display device configured to communicate with the at least one processor of the microneedle therapy device, configured to provide a user interface for receiving setting information about an operation of the microneedle therapy device from the user, and configured to transmit the setting information input by the user to the at least one processor of the microneedle therapy device.
